(19) United States
(12) Patent Application Publication
(10) Pub. No.: US 2020/0324003 A1
(43) Pub. Date: Oct. 15, 2020

(54) UV STERILISER ASSEMBLY AND METHOD OF CONSTRUCTING SAME

(71) Applicant: Alpha-Cure Limited, Daventry (GB)

(72) Inventor: Richard Cherry, Daventry (GB)

(21) Appl. No.: 16/091,278

(22) PCT Filed: Mar. 30, 2017
(86) PCT No.: PCT/GB2017/050891

§ 371 Date: Oct. 4, 2018

(30) Foreign Application Priority Data
Apr. 6, 2016 (GB) 1605798.6

(51) Int. Cl. A61L 2/00; A61L 9/20; C02F 1/32; F21V 7/28

(52) U.S. Cl. CPC A61L 2/0047; A61L 9/20; C02F 1/325; F21V 7/28; C02F 2303/04; A61L 2202/15; C02F 2201/3223; C02F 2201/3228; A61L 2202/11

(57) ABSTRACT

A UV steriliser assembly and associated method for disinfection purposes. The assembly includes a reflector (16) and a UV source, e.g. a lamp (10), configured to emit ultraviolet light at a range of wavelengths. Dependent on the assembly configuration, the reflector (16) is configured to permit or inhibit transmission therethrough of particular UV wavelengths known to assist in the photo-repair of micro-organisms. Transmission of wavelengths known to be destructive to micro-organisms can also be targeted. In this way the effectiveness of the assembly for sterilisation purposes can be optimised.

The present invention relates to a UV steriliser assembly for disinfection purposes, particularly of a type which is capable of an optimised sterilisation effect. The assembly utilises a reflector that targets the most effective wavelengths of light for photochemical breakdown of micro-organisms or specifically remove wavelengths of light that aid in repair of micro-organisms.

### BACKGROUND TO THE INVENTION

In connection with the use of an ultraviolet (UV) light source for disinfection of water, surfaces and/or air it is known that micro-organisms that are deactivated by specific wavelengths of electromagnetic radiation can also undergo a ‘photo-repair’ mechanism when exposed to other wavelengths. In other words, while some wavelengths damage and destroy micro-organisms, other wavelengths can repair those same micro-organisms and encourage growth.

Therefore, especially if a broad spectrum source is used (e.g. from a medium pressure UV lamp) and to a lesser degree narrow spectrum sources (e.g. low pressure germicidal lamp, amalgam lamp or LED UV sources), the overall system efficiency is reduced because wavelengths have conflicting effects on the target organism. Such a feature makes broad spectrum sources in need of optimisation.

GB2531319 describes a UV lamp unit according to a standard industry application. Dichroic reflectors have been used for many years in order to remove heat from a substrate when running UV systems.

### SUMMARY OF THE INVENTION

The present invention seeks to provide a disinfection assembly, particularly of a broad spectrum UV source type, with improved efficiency for sterilisation purposes.

In one broad aspect of the invention there is provided a UV steriliser assembly and method of constructing same according to the appended claims. The assembly is comprised of a UV source, e.g. a lamp, configured to emit ultraviolet light of a range of wavelengths and a reflector associated with the source; wherein the reflector is configured to permit transmission therethrough of identified light wavelengths in order to control/manage/regulate/restrict the wavelength of light reflected by said reflector.

According to the invention, the efficiency of the assembly for sterilisation purposes is increased by removing the wavelengths that play a part in any repair process. The light wavelengths that do fall on the media are restricted to those which actively cause micro-organism damage or have no effect. In an alternative form the identified wavelengths are those known to have the greatest destructive effect on micro-organisms.

In a preferred form the reflector includes a dichroic coating that is formulated according to its light transmission properties for known wavelengths. The reflector/coating is configured to reflect wavelengths that are either damaging or encouraging to micro-organism growth depending on the configuration in relation to the media to be disinfected/sterilised.

As mentioned in the background section above, dichroic reflectors are known to be used in connection with removing heat from a substrate. The present invention utilises a coating specifically for selective removal of UV and visible wavelengths detrimental to micro-organism deactivation in sterilisation systems. The invention is not concerned with removing infrared (heat) and is inspired by a newer understanding of how organisms photo-repair.

In practice, dichroic coatings are applied in a vacuum chamber by evaporating various metals and oxides onto a substrate to form very fine dielectric layers. Each layer is around 2 microns thick and 3 to 50 layers are applied to the surface to build up the dichroic coating.

Each alternate layer is applied with high and low refractive index materials respectively such that as light passes through the interface between them it changes its direction. The amount of direction change is also related to the wavelength of light passing through the various layers. Therefore, by the selection of the different layers and their respective thickness, a skilled person can select which wavelengths can pass through the coating and which will be reflected.

One of the most important properties of dichroic coatings is that they are particularly efficient, with up to 98% of the selected light being reflected. This property, coupled with the nature of the materials used, allows a reflector assembly to operate up to around 400° C., with good chemical inertness and little radiation absorption making them particularly suitable for use in this inventive application.

It is expected that the greatest benefits of the invention will be achieved on broad spectrum UV sources and, as such, the preferred embodiment of assembly utilises a Medium Pressure (MP) lamp, although it is conceivable that other UV sources may be employed and that, in such cases, a dichroic coating could be applied directly to the surface of the UV source.

In practice, performance of the sterilisation assembly may be optimised by a combination of a specially selected dichroic coating and appropriate addition of an element into the lamp chemistry to modify its transmission characteristics, i.e. to desirable wavelengths.

The invention can be applied to a range of lamp layouts and/or associated water sterilisation chambers.

### BRIEF DESCRIPTION OF THE DRAWINGS

FIG. 1 illustrates a graph of the wavelengths of light (nm) emitted from a mercury based lamp;

FIG. 2 illustrates a graph of the wavelengths of light (nm) emitted from a mercury based lamp with a small amount of gallium added;

FIG. 3 illustrates a graph of reflectance where a specific dichroic coating results in UV radiation at a wavelength approximately 250-450 nm being predominantly reflected;

FIG. 4 illustrates a first embodiment of a UV lamp assembly according to the invention;

FIG. 5 illustrates a second embodiment of a UV lamp assembly according to the invention;

FIG. 6 illustrates a third embodiment of a UV lamp assembly according to the invention; and

FIG. 7 illustrates a fourth embodiment of a UV lamp assembly according to the invention.

### DETAILED DESCRIPTION OF PREFERRED EMBODIMENTS ACCORDING TO THE INVENTION

It is usual with MP lamps that the characteristic spectrum comes predominantly from the excitation of mercury in an electric arc and the internal pressure that the lamp is allowed to achieve. It is also common practice to modify the output spectrum with the addition of other chemicals, usually in the form of metals or metal halides. In this way the output of the lamp can be more closely tuned to the specific absorption characteristics that the process requires, in order to be more effective.

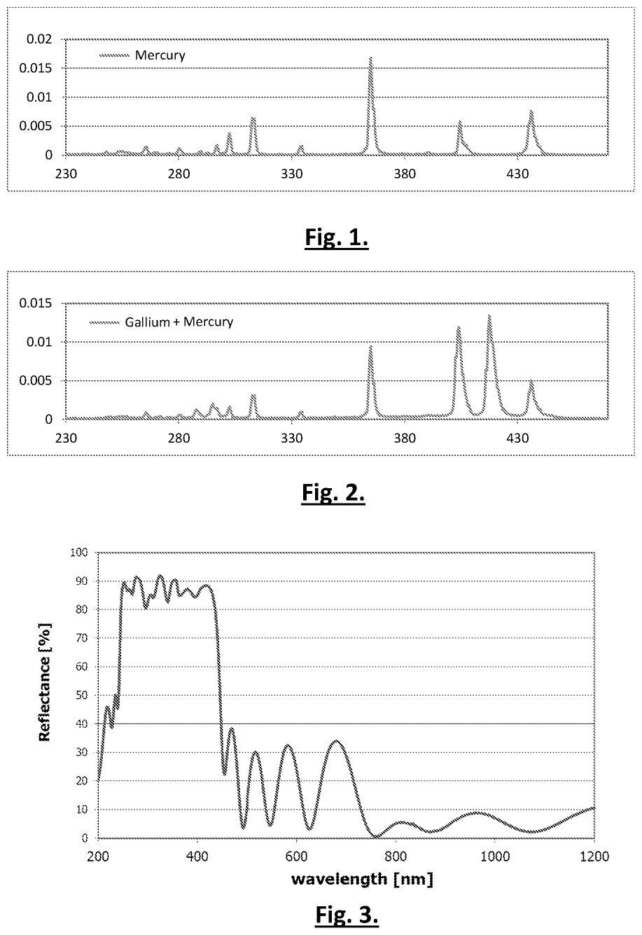

FIGS. 1 and 2 show generic spectrums for MP lamps, the first using mercury only and the second showing, by way of example, the effect of adding small quantities of gallium metal to the mercury. In this example all the common wavelengths are present in both graphs due to the excitation of the mercury but the inclusion of gallium has given an additional peak of 0.135 (relative intensity as measured by a spectrophotometer) at 417 nm. The additional energy to generate this peak has effectively come from shifting energy from the mercury spectrum (it will be noted that other relative peak heights are reduced when comparing FIG. 2 to FIG. 1).

This concept can also be applied to low pressure lamps but, as the operating temperature is much lower, there is less opportunity to use other materials with higher vaporisation temperatures to enhance the spectrum. The spectrum in a low pressure lamp is also of a significantly different shape due to lower operating pressure, and the UV output tends to be concentrated over narrower wavelength ranges.

As is known in the art, lamp output can also be effected by the choice of envelope material. For disinfection purposes this is usually some form of fused silica (e.g. quartz) mainly due to its high transmittance to short wave UV. The addition of doping agents to this material will block certain wavelengths from being emitted, acting as a filter. Conversely the use of highly purified material (e.g. synthetic grades) will allow transmission of shorter wavelengths that would be blocked if a lower grade is used. The invention is enhanced by selecting doping agents that block or inhibit wavelengths associated with photo-repair of microorganisms, since this compliments the coating on the reflector described hereinafter which also targets wavelengths associated with photo-repair.

As most lamps operate above a temperature that a suitable filter media applied directly to a lamp surface could remain serviceable a separate reflector is, in practice, associated with the lamp. Particularly, according to the invention, the reflector can feature a dichroic coating which has the property of being able to be selected and applied in such a way to reflect very specific identifiable wavelengths, with the rest passing through the reflector. FIG. 3 shows how a specific dichroic coating is made to reflect UV radiation from approximately 250-450 nm, but to a large degree permits much of the remaining UV spectrum through, i.e. it is not reflected or absorbed.

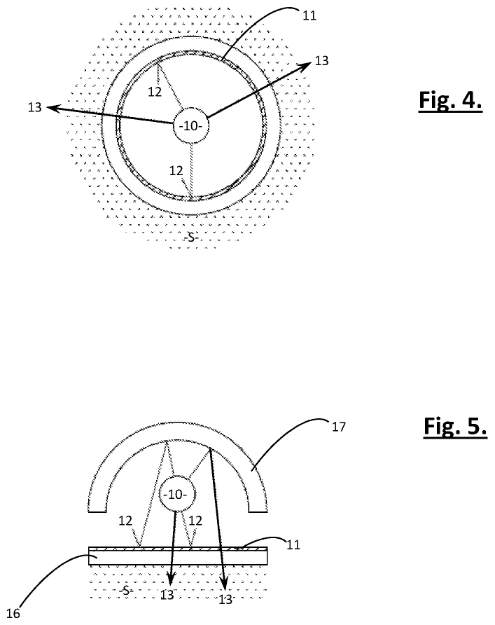

Once the inventive concept, of tailoring a dichroic coating to target wavelengths associated with micro-organism damage and photo-repair, is established it is possible to propose specific mechanical embodiments to carry out the invention. For example, as shown in FIGS. 4 and 5, one mode of operation is to introduce a reflector 11 directly between the light source, lamp 10, and the substrate S to be treated, so that the unwanted wavelengths 12 are effectively rejected and do not pass outside the light source housing. Desirable wavelengths 13, which cause deterioration of micro-organisms for disinfection purposes, ultimately contact/enter the media S (be it water, air or onto a more solid surface) external of the lamp assembly.

FIG. 4 shows a first embodiment where the lamp 10 is mounted and enclosed within a quartz tube 15 that has the coating 11 applied to its surface (or to sectional plates held within a tube if coating of a complete tube is impractical). Such a configuration as illustrated would tend to lend itself to water or air treatment.

Alternatively, FIG. 5 shows the use of a flat reflector 16 to modify the UV output via a coating 11. Such a configuration is particularly applicable to a likewise flat media surface S. The addition of a conventional curved ‘total reflector’ 17 would ensure all of the required wavelengths are allowed to fall on the treated reflector 16 ensuring maximum efficiency. This configuration also lends itself to air and water treatment in addition to solid surfaces.

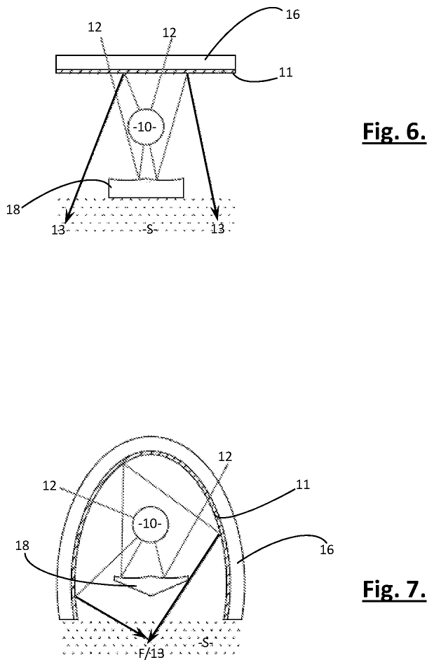

By contrast, if the desirable wavelengths (13) are required to be reflected by the coating 11 into the process media S, then such an arrangement can be achieved by embodiments according to FIGS. 6 and 7. In this case the unwanted wavelengths 12 are rejected by transmission through a reflector.

Referring to FIG. 6, a total reflector 18 located in front of the UV lamp 10 ensures that all radiation is reflected toward a treated reflector 16. Attenuation of the unwanted wavelengths 12 is maximised and the desirable wavelengths 13 are directed toward media S. In the illustrated form reflector 18 is a double concave type which directs light passed and away from lamp 10 toward treated reflector 16.

The embodiment of FIG. 6 can be potentially improved by selecting a suitable shape of reflector. For example, FIG. 7 shows an elliptical treated reflector 16 that serves to focus the output at a point F. This in itself can lead to a higher system efficiency as, although the total energy is the same, the energy density at the point of focus F is vastly increased. Other forms of curved reflector, parabolic or otherwise, can be considered dependent on system requirements.

It can be understood from the foregoing that, by manipulating (i.e. doping) the output characteristic of UV radiation sources combined with the use of dichroic reflectors, the effects of photo-reactivation can be reduced or eliminated in any disinfection system. This will increase system efficiency by reducing the ‘undoing’ effect from the unwanted radiation, which could be turned into a higher micro-organism deactivation rate or energy saving. Particularly, in the past wavelengths in the UV range have been assumed to be destructive and it was visible wavelength light that enabled photo-repair. The present invention recognises the discovery that some UV wavelengths are counterproductive to sterilisation procedures and proposes a novel construction to take advantage of this discovery. Visible wavelength light is also preferably removed, but the improvement of the invention is primarily in reduction of selected UV wavelengths that assist photo-repair.

As micro-organisms have varying resistance to UV treatment, the method and apparatus of the invention allows a high degree of optimisation via customisation, depending on the selected target organisms, by selecting the correct combination of lamp output and reflector characteristics.

Additionally, energy density benefits can be achieved in a suitably designed system that has the ability to focus a “tuned” UV output on to the media being treated. High energy density could also be used to increase efficiency in the UV breakdown of other non-biological chemicals (e.g. hormones or nitrates that exist in water supplies).

The invention is exemplified by a tailored dichroic coating selected to permit or inhibit UV wavelengths associated with photo repair, but it is conceivable that other treatments or techniques could be applied to the reflector to achieve equivalent results.

In principle, the general concept of the invention can be adapted for other photochemical processes. For example, provision of a coating not necessarily for the destruction of microorganisms (by removal of repairing wavelengths) but to enhance or inhibit some other quality.

1. A UV steriliser assembly comprised of:
a UV source configured to emit ultraviolet light; and
a reflector associated with the UV source;
wherein the reflector is configured to permit or inhibit transmission therethrough of selected wavelengths of the ultraviolet light known to assist in the photo-repair of micro-organisms.

2. The UV steriliser assembly of claim 1 wherein the reflector is further configured to permit or inhibit transmission therethrough of selected wavelengths of the ultraviolet light known to be destructive to micro-organisms or neutral.

3. The UV steriliser assembly of claim 1 wherein the reflector includes a dichroic coating formulated according to its light transmission properties for the selected wavelengths.

4. (canceled)

5. The UV steriliser assembly of claim 1 wherein the reflector is further configured to permit or inhibit transmission therethrough of visible light.

6. The UV steriliser assembly of claim 1 wherein the UV source is a Medium Pressure (MP) lamp.

7. The UV steriliser assembly of claim 1 wherein the lamp is configured to excite mercury for producing a broad UV spectrum.

8. The UV steriliser assembly of claim 1 wherein the UV source is doped to tune the ultraviolet light to remove or inhibit wavelengths known to assist in the photo-repair of micro-organisms.

9. The UV steriliser assembly of claim 1 wherein the reflector is located adjacent or comprises an external wall of the steriliser assembly that, in use, is between the UV source and a media which is to be treated, and wherein the reflector is configured to inhibit transmission therethrough of selected wavelengths of the ultraviolet light known to assist in the photo-repair of micro-organisms.

10. The UV steriliser assembly of claim 9 wherein the reflector is formed on the surface of a tube within which the UV source is mounted.

11. The UV steriliser assembly of claim 1 wherein a total reflector is located opposite the reflector and wherein the reflector is configured to permit transmission therethrough of selected wavelengths of the ultraviolet light known to assist in the photo-repair of micro-organisms.

12. The UV steriliser assembly of claim 11 wherein either the reflector or the total reflector is elliptical.

13. The UV steriliser assembly of claim 11 wherein either the reflector or the total reflector is flat.

14. The UV steriliser assembly of claim 11 wherein either the reflector or the total reflector is concave to direct light away from the lamp.

15. A disinfection method including:
provision of a reflector with a coating or composition that permits or inhibits transmission therethrough of selected wavelengths of UV light known to assist in the photo-repair of micro-organisms;
arranging the reflector in combination with a broad spectrum UV source.

16. The disinfection method of claim 15 wherein a media to be treated by the method is either located in front of the reflector, when selected wavelengths are permitted; or behind the reflector, when selected wavelengths are inhibited.

17. The disinfection method of claim 15 wherein the coating or composition is a dichroic coating.

18. The disinfection method of claim 15 wherein the coating or composition also permits or inhibits transmission therethrough of selected wavelengths of UV light known to be destructive to micro-organisms.

19. The disinfection method of claim 15 wherein the reflector is formed on the surface of a tube within which the UV source is mounted, and wherein the reflector is configured to inhibit transmission therethrough of selected wavelengths of the ultraviolet light known to assist in the photo-repair of micro-organisms.

20. The disinfection method of claim 15 wherein a total reflector is arranged opposite the reflector, and wherein the reflector is configured to permit transmission therethrough of selected wavelengths of the ultraviolet light known to assist in the photo-repair of micro-organisms.

21. (canceled)

22. (canceled)

23. (canceled)

24. (canceled)

25. A UV light assembly comprised of:
a UV source configured to emit ultraviolet light of a range of wavelengths; and
a reflector associated with the UV source;
wherein the reflector is configured to permit or inhibit transmission therethrough of targeted wavelengths of light associated with a photo-chemical process.
